(19) United States
(12) Patent Application Publication
(10) Pub. No.: US 2022/0088037 A1
(43) Pub. Date: Mar. 24, 2022

(54) A SUSTAINED RELEASE COMPOSITION COMPRISING A HYDROXYALKYL METHYLCELLULOSE

(71) Applicant: DDP SPECIALTY ELECTRONIC MATERIALS US, INC., Collegeville, PA (US)

(72) Inventor: Oliver Petermann, BOMLITZ (DE)

(21) Appl. No.: 17/312,417

(22) PCT Filed: Dec. 17, 2019
(86) PCT No.: PCT/US2019/066728

§ 371 Date: Jun. 10, 2021

(30) Foreign Application Priority Data
Dec. 18, 2018 (EP) 18213429.6

(51) Int. Cl. A61K 31/616; A61K 9/16; A61K 31/155; A61K 31/167; A61K 9/00

(52) U.S. Cl. CPC A61K 31/616; A61K 9/1652; A61K 9/0053; A61K 31/167; A61K 31/155

(57) ABSTRACT

A sustained release composition for oral administration comprises particles of a physiologically active ingredient mixed with a hydroxyalkyl methylcellulose, wherein the ether substituents are methyl groups, hydroxyalkyl groups, and optionally alkyl groups being different from methyl,
the hydroxyalkyl methylcellulose has an MS(hydroxyalkyl) of 0.05 to 1.00, and hydroxy groups of anhydroglucose units are substituted with methyl groups such that [s23/s26−0.2*MS(hydroxyalkyl)] is 0.30 or less, and
wherein the concentration of hydroxyalkyl methylcellulose is 0.1-10% by dry weight of the active ingredient.

### FIELD

The present invention relates to novel sustained release compositions comprising a physiologically active ingredient and a hydroxyalkyl methylcellulose.

### INTRODUCTION

Sustained release dosage forms have found wide application in a variety of technology areas such as in personal care and agricultural applications, water treatment and in particular pharmaceutical applications. Sustained release dosage forms are designed to release a finite quantity of an active ingredient into an aqueous environment over an extended period of time. Sustained release pharmaceutical dosage forms are desirable because they provide a method of delivering a long-lasting dose in a single application without overdosing. Known sustained release pharmaceutical dosage forms contain a drug or a vitamin whose release is controlled by a polymeric matrix which, for instance, may comprise one or more water-soluble cellulose ethers. Water-soluble cellulose ethers hydrate on the surface of a tablet to form a gel layer. A fast formation of the gel layer is important to prevent wetting of the interior and disintegration of the tablet core. Once the gel layer is formed, it controls the penetration of additional water into the tablet. As the outer layer fully hydrates and dissolves, an inner layer must replace it and be sufficiently cohesive and continuous to retard the influx of water and control drug diffusion.

A commonly used cellulose ether for providing sustained release of an active ingredient from an oral dosage form is hydroxypropyl methylcellulose (HPMC). For instance, U.S. Pat. No. 4,734,285 discloses that the release of an active ingredient can be prolonged by employing a fine particle sized HPMC as an excipient in a solid tablet. HPMC is used in commercial oral pharmaceutical formulations as a component of a polymeric matrix providing sustained release of a drug usually at a concentration of 30% to 60% by weight of the oral dosage form.

It is a well-known problem in the pharmaceutical art that some patients, especially children or the elderly, or patients with dysphagia, find it difficult to swallow conventional oral dosage forms such as capsules or tablets. In particular, this is the case if the drug administered in the dosage form is a highly dosed drug which, when the drug is formulated with pharmaceutical excipients in the typical amounts included in commercial dosage forms, either makes each dosage form very large or requires the dose to be divided among two or more dosage forms that have to be swallowed at the same time.

It would therefore be desirable to develop an oral dosage form where a drug is formulated with a reduced amount of excipient(s) to permit a reduction in the overall size of the dosage form and improve the swallowability without compromising the sustained release properties thereof.

### SUMMARY OF THE INVENTION

It has surprisingly been found that when a hydroxyalkyl methylcellulose with a specific substitution pattern is used as an excipient in admixture with a physiologically active ingredient, it is capable of forming a stable hydrogel in an aqueous environment at body temperature (i.e. about 37° C.) and provide sustained release of the active ingredient. This is the case even when it is used at much lower concentrations that the concentrations of HPMC used in commercial formulations.

Accordingly, the present invention relates to a sustained release composition for oral administration comprising particles of a physiologically active ingredient mixed with a hydroxyalkyl methylcellulose, wherein the ether substituents are methyl groups, hydroxyalkyl groups, and optionally alkyl groups being different from methyl,
the hydroxyalkyl methylcellulose has an MS(hydroxyalkyl) of 0.05 to 1.00, and
hydroxy groups of anhydroglucose units are substituted with methyl groups such that [s23/s26−0.2*MS(hydroxyalkyl)] is 0.30 or less,

wherein s23 is the molar fraction of anhydroglucose units wherein only the two hydroxy groups in the 2- and 3-positions of the anhydroglucose unit are substituted with methyl groups,

wherein s26 is the molar fraction of anhydroglucose units wherein only the two hydroxy groups in the 2- and 6-positions of the anhydroglucose unit are substituted with methyl groups, and

wherein the concentration of hydroxyalkyl methylcellulose is 0.1-10% by dry weight of the active ingredient.

It has surprisingly been found that hydroxyalkyl methylcellulose ethers where [s23/s26−0.2*MS(hydroxyalkyl)] is 0.30 or less are capable of forming hydrogels at low concentrations at body temperature which makes them particularly useful as an excipient for a sustained release dosage form which means that it has the function to regulate the release of an active ingredient from the dosage form over an extended period of time. For the present purpose, these hydroxyalkyl methylcelluloses are termed “G” (for gelling) hydroxyalkyl (hydroxypropyl or hydroxyethyl) methylcellulose in the following (abbreviated to G-HPMC in the examples). By way of contrast, commercial HPMC grades such as METHOCEL™ K4M and METHOCEL™ E4M where [s23/s26−0.2*MS(hydroxyalkyl)] is more than 0.30 precipitate at low concentrations and do not form hydrogels at body temperature.

EP 2627676 B1 discloses hydroxyalkyl methylcellulose with an MS(hydroxyalkyl) of 0.05 to 1.00, and [s23/s26−0.2*MS(hydroxyalkyl)] of 0.30 or less. The hydroxyalkyl methylcellulose is proposed for production of capsules and coatings for dosage forms. It is not suggested that the hydroxyalkyl methylcellulose can be used as a polymeric matrix material in a very low concentration in a solid dosage form compared to the concentration of the active ingredient while retaining its sustained release properties.

### BRIEF DESCRIPTION OF THE DRAWINGS

FIG. 1 is a graph showing the release over time of acetaminophen (APAP) from a composition of the invention containing a 3% solution of hydrogel forming hydroxypropyl methylcellulose as described herein (G-HPMC) when a gelatin capsule containing the composition and dried overnight at 50° C. is immersed in 900 ml of 0.1 N HCl pH 1.1.

FIG. 2 is a graph showing the release over time of acetaminophen (APAP) from a composition containing a 3% solution of METHOCEL K4M HPMC when a gelatin capsule containing the composition is immersed in 900 ml of 0.1 N HCl pH 1.1. Release from a wet capsule is shown as -♦-, and release from a capsule dried overnight at 50° C. is shown as -▪-.

FIG. 3 is a graph showing the release over time of acetaminophen (APAP) from a gelatin capsule containing a composition containing a 3% solution of METHOCEL E4M HPMC immersed in 900 ml of 0.1 N HCl pH 1.1. Release from a wet capsule is shown as -♦-, and release from a capsule dried overnight at 50° C. is shown as -▪-.

### DESCRIPTION OF EMBODIMENTS

In the G hydroxyalkyl methylcellulose ethers used as a sustained release matrix material in the present composition the ether substituents are methyl groups, hydroxyalkyl groups, and optionally alkyl groups which are different from methyl.

The hydroxyalkyl groups can be the same or different from each other. Preferably the hydroxyalkyl methylcellulose comprises one or two kinds of hydroxyalkyl groups, more preferably one or more kinds of hydroxy-C₁₋₃-alkyl groups, such as hydroxypropyl and/or hydroxyethyl. Useful optional alkyl groups are, e.g., ethyl or propyl, ethyl being preferred. Preferred ternary cellulose ethers are ethyl hydroxypropyl methylcelluloses, ethyl hydroxyethyl methylcelluloses, or hydroxyethyl hydroxypropyl methylcelluloses. Preferred G hydroxyalkyl methylcelluloses are G hydroxypropyl methylcelluloses (G-HPMC) or G hydroxyethyl methylcelluloses (G-HEMC), G hydroxypropyl methylcellulose being particularly preferred.

An essential feature of the G hydroxyalkyl methylcellulose ethers is their unique distribution of methyl groups on the anhydroglucose units such that [s23/s26−0.2*MS(hydroxyalkyl)] is 0.30 or less, preferably 0.27 or less, more preferably 0.25 or less, such as 0.23 or less. Typically [s23/s26−0.2*MS(hydroxyalkyl)] is 0.07 or more, more typically 0.10 or more, and most typically 0.13 or more. More specifically, in the case of hydroxyethyl methylcelluloses the upper limit for [s23/s26−0.2*MS(hydroxyalkyl)] is 0.30 and preferably 0.27. In the case of hydroxypropyl methylcelluloses the upper limit for [s23/s26−0.2*MS(hydroxyalkyl)] is 0.30, preferably 0.27; more preferably 0.25 and most preferably 0.23. Hydroxyalkyl methylcelluloses with this substitution pattern have been found to have a low dissolution temperature, such as a dissolution temperature of 10° C. or less, whereas hydroxyalkyl methylcelluloses with a s23/s26 ratio of more than 0.30 dissolve at room temperature (21-25° C.). Due to the low dissolution temperature, G-hydroxyalkyl methylcellulose do not dissolve at body temperature, but nevertheless form a hydrogel in aqueous liquids at body temperature when formulated with a drug substance, possibly due to swelling.

As used herein, the symbol “*” represents the multiplication operator. In the ratio s23/s26, s23 is the molar fraction of anhydroglucose units wherein only the two hydroxy groups in the 2- and 3-positions of the anhydroglucose unit are substituted with methyl groups and s26 is the molar fraction of anhydroglucose units wherein only the two hydroxy groups in the 2- and 6-positions of the anhydroglucose unit are substituted with methyl groups. For determining the s23, the term “the molar fraction of anhydroglucose units wherein only the two hydroxy groups in the 2- and 3-positions of the anhydroglucose unit are substituted with methyl groups” means that the 6-positions are not substituted with methyl; for example, they can be unsubstituted hydroxy groups or they can be substituted with hydroxyalkyl groups, methylated hydroxyalkyl groups, alkyl groups different from methyl or alkylated hydroxyalkyl groups. For determining the s26, the term “the molar fraction of anhydroglucose units wherein only the two hydroxy groups in the 2- and 6-positions of the anhydroglucose unit are substituted with methyl groups” means that the 3-positions are not substituted with methyl; for example, they can be unsubstituted hydroxy groups or they can be substituted with hydroxyalkyl groups, methylated hydroxyalkyl groups, alkyl groups different from methyl or alkylated hydroxyalkyl groups.

Formula I below illustrates the numbering of the hydroxy groups in anhydroglucose units. Formula I is only used for illustrative purposes and does not represent the present G hydroxyalkyl methylcellulose; the substitution with hydroxyalkyl groups is not shown in Formula I.

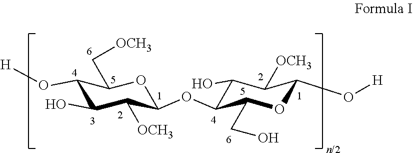

The G hydroxyalkyl methylcellulose preferably has a DS(methyl) of from 1.2 to 2.2, more preferably from 1.25 to 2.10, and most preferably from 1.40 to 2.00. The degree of the methyl substitution, DS(methyl), of a cellulose ether is the average number of OH groups substituted with methyl groups per anhydroglucose unit. For determining the DS(methyl), the term “OH groups substituted with methyl groups” does not only include the methylated OH groups at the polymer backbone, i.e., that are directly a part of the anhydroglucose unit, but also methylated OH groups that have been formed after hydroxyalkylation.

The G hydroxyalkyl methylcellulose has an MS(hydroxyalkyl) of 0.05 to 1.00, preferably 0.07 to 0.80, more preferably 0.08 to 0.70, most preferably 0.10 to 0.60, and particularly 0.10 to 0.50. The degree of the hydroxyalkyl substitution is described by the MS (molar substitution). The MS(hydroxyalkyl) is the average number of hydroxyalkyl groups which are bound by an ether bond per mole of anhydroglucose unit. During the hydroxyalkylation, multiple substitutions can result in side chains.

The determination of the % methoxyl and % hydroxypropoxyl in hydroxypropyl methylcellulose is carried out according to the United States Pharmacopeia (USP 32). The values obtained are % methoxyl and % hydroxypropoxyl. These are subsequently converted into degree of substitution (DS) for methyl substituents and molar substitution (MS) for hydroxypropyl substituents. Residual amounts of salt have been taken into account in the conversion. The DS(methyl) and MS(hydroxyethyl) in hydroxyethyl methylcellulose is effected by Zeisel cleavage with hydrogen iodide followed by gas chromatography. (G. Bartelmus and R. Ketterer, Z. Anal. Chem. 286 (1977) 161-190).

Methods of making hydroxyalkyl methylcellulose are described in detail in EP 2627676 B1 which is hereby incorporated by reference. Some aspects of the process for making hydroxyalkyl methylcellulose are described in more general terms below.

Generally speaking, cellulose pulp or, as the reaction of cellulose pulp to the hydroxyalkyl methyl cellulose proceeds, to partially reacted cellulose pulp, is alkalized in two or more stages, preferably in two or three stages, in one or more reactors with an aqueous alkaline solution of an alkali metal hydroxide, more preferably sodium hydroxide. The aqueous alkaline solution preferably has an alkali metal hydroxide content of from 30 to 70 percent, more preferably from 35 to 60 percent, most preferably from 48 to 52 percent, based on the total weight of the aqueous alkaline solution.

In one embodiment, an organic solvent such as dimethyl ether is added to the reactor as a diluent and a coolant. Likewise, the headspace of the reactor is optionally purged with an inert gas (such as nitrogen) to control oxygen-catalyzed depolymerization of the cellulose ether product.

Typically, from 1.2 to 2.0 molar equivalents of alkali metal hydroxide per mole of anhydroglucose units in the cellulose are added in the first stage. Uniform swelling and distribution in the pulp is optionally controlled by mixing and agitation. In the first stage the rate of addition of the alkali metal hydroxide agent is not very critical. It can be added in several portions, e.g., in 2 to 4 portions, or continuously. The temperature at the first stage of contacting the alkali metal hydroxide with the cellulose pulp is typically maintained at or below about 45° C. The first stage of alkalization typically lasts from 15 to 60 minutes.

A methylating agent, such as methyl chloride or dimethyl sulfate is also added to the cellulose pulp, typically after the addition of the alkali metal hydroxide. The total amount of the methylating agent is generally from 2 to 5.3 mols per mole of anhydroglucose units. The methylating agent can be added to the cellulose or, as the reaction of cellulose pulp to the hydroxyalkyl methyl cellulose proceeds, to partially reacted cellulose pulp, in a single stage, but it is preferably added in two or more stages, more preferably two or three stages, most preferably two stages.

If the methylating agent is added in a single stage, it is generally added in an amount of from 3.5 to 5.3 moles of methylating agent per mole of anhydroglucose units, but in any event it is added in at least an equimolar amount, compared to the added total molar amount of alkali metal hydroxide, before heating the reaction mixture. If the methylating agent is added in a single stage, it is preferably added at a rate of from 0.25 to 0.5 molar equivalents of methylating agent per mole of anhydroglucose units per minute.

If the methylating agent is added in two stages, in the first stage it is generally added in an amount of from 2 to 2.5 moles of methylating agent per mole of anhydroglucose units before heating the reaction mixture, but in any event it is added in at least an equimolar amount, compared to the molar amount of alkali metal hydroxide added in the first stage of alkali metal hydroxide addition. If the methylating agent is added in two stages, the methylating agent of the first stage is preferably added at a rate of from, 0.25 to 0.5 molar equivalents of methylating agent per mole of anhydroglucose units per minute. The methylating agent of the single stage or of the first stage may be pre-mixed with the suspending agent. In this case the mixture of suspending agent and methylating agent preferably comprises from 20 to 50 weight percent, more preferably from 30 to 50 weight percent, of the suspending agent, based on the total weight of methylating agent and suspending agent. Once the cellulose has been contacted with the alkali metal hydroxide and methylating agent, the reaction temperature is typically increased over a time period of 30 to 80 minutes, more typically of 50 to 70 minutes, to a temperature of about 70-85° C., preferably about 75-80° C., and reacted at this temperature for 10 to 30 minutes.

If the methylating agent is added in two stages, the second stage of methylating agent is generally added to the reaction mixture after having heated the reaction mixture to a temperature of about 70-85° C. for 10 to 30 minutes. The methylating agent of second stage is generally added in an amount of from 1.5 to 3.4 moles per mole of anhydroglucose units, but in any event it is added in at least an equimolar amount, compared to the molar amount of alkali metal hydroxide present in the reaction mixture. Accordingly, the methylating agent of the second stage, if any, is added to the reaction mixture before or during the second and optionally third stage of alkali metal hydroxide addition in such a manner that the alkali metal hydroxide is not contacted in excess amounts with the cellulose pulp. The methylating agent of the second stage is preferably added at a rate of from 0.25 to 0.5 molar equivalents of methylating agent per mole of anhydroglucose units per minute. If the methylating agent is added in two stages, the molar ratio between the methylating agent of the first stage and the methylating agent of the second stage is generally from 0.68:1 to 1.33:1.

If the alkali metal hydroxide is added in two stages, typically from 1.0 to 2.9 molar equivalents of alkali metal hydroxide per mole of anhydroglucose units are added in the second stage, after the addition of the methylating agent of the single stage or first stage and simultaneously with or after the addition of the methylating agent of the second stage, if any. The molar ratio between the alkali metal hydroxide of the first stage and the alkali metal hydroxide of the second stage generally is from 0.6:1 to 1.2:1. It is important to add the alkali metal hydroxide used in the second stage slowly, i.e., at a rate of less than 0.04, typically at a rate of less than 0.03 molar equivalents of alkali metal hydroxide per mole of anhydroglucose units per minute. The alkali metal hydroxide of the second stage is generally added at a temperature of from 55 to 80° C., preferably from 60 to 80° C.

As an alternative to the procedure above wherein the methylating agent and alkali metal hydroxide each are added in two stages, the methylating agent of the second stage is added to the reaction mixture after a portion of the alkali metal hydroxide of the second stage has been added, followed by subsequent addition of alkali metal hydroxide; i.e., the methylating agent is added in a second stage, which is followed by the addition of a third stage of alkali metal hydroxide. In this embodiment of the process, the total amount of alkali metal hydroxide per mole of anhydroglucose added in the second and third stage is generally 1.0 to 2.9 moles per mole of anhydroglucose units, of which preferably 40 to 60 percent are added in the second stage and 60 to 40 percent are added in the third stage. Preferably the alkali metal hydroxide used in the third stage is added slowly, i.e., at a rate of less than 0.04, typically at a rate of less than 0.03 molar equivalents of alkali metal hydroxide per mole of anhydroglucose units per minute. The methylating agent and alkali metal hydroxide of the third stage are generally added at a temperature of from 55 to 80° C., preferably from 65 to 80° C.

One or more, preferably one or two, hydroxyalkylating agents, such as ethylene oxide and/or propylene oxide are also added to the cellulose pulp, or, as the reaction of cellulose pulp to the hydroxyalkyl methyl cellulose proceeds, to partially reacted cellulose pulp, either before, after, or concurrently with the alkali metal hydroxide added in the first stage. Preferably only one hydroxyalkylating agent is used. The hydroxyalkylating agent is generally added in an amount of 0.2 to 2.0 mole of hydroxyalkylating agent per mole of anhydroglucose units. The hydroxyalkylating agent is advantageously added before heating the reaction mixture to the reaction temperature, i.e. at a temperature of from 30 to 80° C., preferably from 45 to 80° C.

An additional alkylating agent, different from a methylating agent, may also be added to the cellulose pulp, either before, after, or concurrently with the alkali metal hydroxide added in the first stage. A useful alkylating agent is an ethylating agent, such as ethyl chloride. The additional alkylating agent is generally added in an amount of 0.5 to 6 moles of alkylating agent per mole of anhydroglucose units. The alkylating agent is advantageously added before heating the reaction mixture to the reaction temperature, i.e. at a temperature of from 30 to 80° C., preferably from 45 to 80° C.

The hydroxyalkyl methylcellulose is washed to remove salt and other reaction by-products. Any solvent in which salt is soluble may be employed, but water is preferred. The hydroxyalkyl methylcellulose may be washed in the reactor, but is preferably washed in a separate washer located downstream of the reactor. Before or after washing, the hydroxyalkyl methylcellulose may be stripped by exposure to steam to reduce residual organic content.

The hydroxyalkyl methylcellulose is dried to a reduced moisture and volatile content of preferably about 0.5 to about 10.0 weight percent water and more preferably about 0.8 to about 5.0 weight percent water and volatiles, based upon the sum of the weight of hydroxyalkyl methylcellulose and the volatiles. The reduced moisture and volatiles content enables the hydroxyalkyl methylcellulose to be milled into particulate form. The hydroxyalkyl methylcellulose is milled to particulates of desired size. If desired, drying and milling may be carried out simultaneously.

According to the above-mentioned process a hydroxyalkyl methylcellulose is obtained which generally has a viscosity of more than 150 mPa·s, preferably from 500 to 200,000 mPa·s, more preferably from 500 to 100,000 mPa·s, most preferably from 1,000 to 80,000, particularly from 1,000 to 60,000, determined in a 1.5% by weight aqueous solution at 20° C. in a Haake RS600 at a shear rate of 2.55 s⁻¹.

It has been found that the present G hydroxyalkyl methylcelluloses which have a viscosity of more than 150 mPa·s, determined in a 1.5% by weight aqueous solution at 20° C. and a shear rate of 2.55 s⁻¹ as defined above have a high gel strength. When an aqueous solution of the G hydroxyalkyl methylcellulose is characterized by G′/G″≥1, i.e. when it forms a gel, the gel strength is measured as storage modulus G′. G hydroxyalkyl methylcelluloses which have a viscosity of more than 150 mPa·s, determined in a 1.5% by weight aqueous solution at 20° C. and a shear rate of 2.55 s⁻¹, generally have a storage modulus G′ of at least 50 Pa, preferably at least 100 Pa, more preferably at least 150, and most preferably at least 200 Pa, measured as a 1.5 weight percent aqueous solution at 80° C. Such a storage modulus G′ is generally even achieved when the MS(hydroxyalkyl) is within the range of >0.30 and up to 1.00, more typically up to 0.80, most typically up to 0.60. When the MS(hydroxyalkyl) is within the range of 0.05 to 0.30, the present hydroxyalkyl methylcellulose generally has a storage modulus G′ of at least 100 Pa, preferably of at least 150 Pa, more preferably at least 200 Pa, most preferably at least 250 Pa, and in many cases even at least 300 Pa, measured as a 1.5 weight percent aqueous solution at 80° C. Under optimized conditions storage moduli of up to 20,000 Pa, typically of up to 10,000 Pa, and more typically of up to 5,000 Pa, measured as a 1.5 weight percent aqueous solution at 80° C. can be achieved. The gel strength of the G hydroxyalkyl methylcelluloses, which have a viscosity of more than 150 mPa·s, determined in a 1.5% by weight aqueous solution at 20° C. and a shear rate of 2.55 s⁻¹ as defined above, is higher than the gel strength of comparative cellulose ethers having a comparable viscosity and types and percentages of substitution. This makes them highly advantageous as polymeric matrix material for producing solid sustained release dosage forms.

In another aspect, the viscosity of the hydroxyalkyl methylcellulose is typically from 2 to 200 mPa·s, preferably from 2 to 100 mPa·s, more preferably from 2.5 to 50 mPa·s, in particular from 3 to 30 mPa·s, measured as a 2 weight-% aqueous solution at 20° C. according to ASTM D2363-79 (Reapproved 2006). Such low viscosity hydroxyalkyl methylcellulose is particularly useful for the manufacture of solid dosage forms in which an active ingredient is embedded in a matrix of the polymer. Such hydroxyalkyl methylcellulose has a lower gelation temperature in aqueous solutions than known hydroxyalkyl methylcelluloses of the same viscosity and concentration in aqueous solutions. The gelation temperature depends on the MS(hydroxyalkyl). It has been found that a 20% by weight aqueous solution of the low viscosity G hydroxyalkyl methylcellulose, such as low viscosity G hydroxypropyl methylcellulose or low viscosity G hydroxyethyl methylcellulose, generally meets the relationship

[(gelation temperature [° C.]/1[° C.])−(150*MS(hydroxyalkyl)]<20, preferably <10, more preferably <0, and most preferably <−5, wherein the gelation temperature is the temperature in ° C. at which G′/G″=1, G′ being the storage modulus and G″ being the loss modulus of the 20% by weight aqueous solution of the cellulose ether. No precipitation is detected when heating a 20% by weight aqueous solution of such low viscosity cellulose ether of the present invention to cause gelation of the aqueous solution.

The storage modulus G′, the loss modulus G″ and the gelation temperature at which G′/G″=1, each of a 20% by weight aqueous solution of the cellulose ether are measured in a temperature sweep experiment using an Anton Paar Physica MCR 501 with a peltier temperature control system in oscillation shear flow. A parallel plate (PP-50) geometry with a measurement gap of 1 mm is used. The geometry is covered with a metal ring (inner diameter of 65 mm, width of 5 mm, and height of 15 mm) around the geometry and the outer surface of the solution is covered with paraffin oil. The measurements are performed at a constant frequency of 2 Hz. and a constant strain (deformation amplitude) of 0.5% from 5° C. to 75° C. or −2° C. to 75° C., if 5° C. is already near to the cross-over of G′ and G″. These measurements are conducted with a heating rate of 1° C./min with a data collection rate of 4 points/min. The storage modulus G′, which is obtained from the oscillation measurements, represents the elastic properties of the solution. The loss modulus G″, which is obtained from the oscillation measurements, represents the viscous properties of the solution. During the gelation process of the sample, G′ exceeds G″. The cross-over of G′ and G″ represents the gelation temperature. Some cellulose ethers of the present invention might show two points of cross-over of G′ and G″. In such case the gelation temperature is the temperature at which G′/G″=1 and G″>G′ at a temperature which is 1° C. colder than G′/G″=1.

A G hydroxyalkyl methylcellulose with low viscosity may suitably be prepared by a partial depolymerization process. Partial depolymerization processes are well known in the art and described, for example, in European Patent Applications EP 1,141,029; EP 210,917; EP 1,423,433; and U.S. Pat. No. 4,316,982. Alternatively, partial depolymerization can be achieved during the production of the G hydroxyalkyl methylcellulose, for example by the presence of oxygen or an oxidizing agent. In such partial depolymerization process a G hydroxyalkyl methylcellulose can be obtained which has a viscosity of from 2 to 200 mPa·s, preferably from 2 to 100 mPa·s, more preferably from 2.5 to 50 mPa·s, in particular from 3 to 30 mPa·s, determined in a 2% by weight aqueous solution at 20° C. according to ASTM D2363-79 (Reapproved 2006).

The G hydroxyalkyl methylcellulose is useful as an excipient for a sustained release dosage form which means that is has the function to regulate the release of an active ingredient from the dosage form over an extended period of time. The term “sustained release” is used herein synonymously with the term “controlled release”. Sustained release is an approach by which active ingredients such as physiologically active compounds are made available at a rate and duration designed to accomplish an intended effect. The G hydroxypropyl methylcellulose is useful for forming all or part of a polymeric matrix in which the active ingredient is embedded. The polymeric matrix may additionally comprise one or more other polymers capable of providing sustained release of the active ingredient from the dosage form. The G hydroxyalkyl methylcellulose typically constitutes at least 50%, preferably 60-100%, more preferably 70-100%, even more preferably 80-100%, and most preferably 90-100% by weight of the polymeric matrix. When one or more other polymers are included in the polymeric matrix, they may be selected from cellulose ethers such as methylcellulose, hydroxyethyl methylcellulose, hydroxypropyl cellulose or carboxymethylcellulose, or they may be selected from other polysaccharides such as sodium alginate or calcium alginate.

The G hydroxyalkyl methylcellulose may be included in sustained release dosage forms, in particular dosage forms intended for oral administration of drugs or other physiologically active ingredients and release thereof into the gastrointestinal tract so as to control the absorption rate of the active ingredient to achieve a desired blood plasma profile. The combined amount of G hydroxyalkyl methylcellulose and active ingredient in the dosage form is preferably at least 50%, more preferably at least 70% and most preferably at least 90% by dry weight of the dosage form, and preferably up to 100%, more preferably up to 98% and most preferably up to 95% by dry weight of the dosage form. The dosage form is designed to provide a constant or nearly constant level of the active ingredient in plasma with reduced fluctuation via a slow, continuous release of the active ingredient over an extended period of time such as a period of between 4 and 30 hours, preferably between 8 and 24 hours to release all or almost all of the active ingredient from the dosage form.

It has been found that sustained release dosage forms such as tablets and capsules wherein the polymer matrix is formed partially or completely from G hydroxyalkyl methylcellulose remains intact over an extended time period such as at least 4 hours, preferably at least 6 hours and under optimized conditions at least 8 hours. Without wanting to be bound by theory, it is believed that the G hydroxyalkyl methylcellulose is hydrated to form a strong swollen layer on the outer surface of the dosage form upon contact with an aqueous liquid at body temperature. The strong swollen layer minimizes the release of the active ingredient caused by erosion of the dosage form. Since the tablets or capsule contents do not disintegrate (i.e. do not fall apart to any significant degree), the release of the active ingredient is controlled by the slow diffusion from the swollen layer that has been formed by hydration of the G hydroxyalkyl methylcellulose on the outer surface of the dosage form. A strong swollen layer reduces the penetration of water into the sustained release dosage form, which delays the release of the active ingredient, particularly a water-soluble active ingredient, into an aqueous environment due to a reduced amount of water in the zone of the dosage form into which water diffuses and dissolves the active ingredient.

While the concentration of G hydroxyalkyl methylcellulose in the composition may vary between wide limits, it has surprisingly been found that essentially the same rate of release of the active ingredient can be achieved when a much lower amount of G hydroxyalkyl methylcellulose is included as all or part of the polymeric matrix. Thus, it has been found that an acceptable rate of release of the active ingredient can be achieved compared to commercial sustained release dosage forms that typically contain about 30% by weight of HPMC when the G hydroxyalkyl methylcellulose is included in the dosage form as the sole matrix polymer in a concentration of 0.1-10%, preferably 0.2-5.0%, more preferably 0.5-4.0%, more preferably 0.75-2.0% and still more preferably 1-1.8% by dry weight of the active ingredient. In one embodiment, the G hydroxyalkyl methylcellulose is included in the dosage form as the sole matrix polymer in a concentration of about 1.5% by dry weight of the active ingredient. The resulting sustained release dosage form, such as tablet or capsule, is smaller in size and therefore easier to ingest. It has furthermore been found that a satisfactory release rate may be obtained without adding any other excipients to the dosage form, though a surfactant may optionally be added during the manufacturing process as a defoaming agent.

In an embodiment, the composition comprises an additive which on ingestion reacts with gastric fluid to generate a gas such as CO₂. The developing gas is trapped in the hydrogel which, as a result, floats to the surface of the gastric contents resulting in prolonged gastric retention time. The prolonged gastric retention time improves the bioavailability of the active ingredient, increases the duration of release and improves the solubility of active ingredients that are not readily soluble in the high pH environment of the intestine. Examples of additives which generate gas in contact with gastric fluid are alkali metal and alkaline earth metal salts such as CaCO₃ and Na₂CO₃. The concentration of the additive may be in the range of 1-5% by weight, preferably 1.5-3% by weight, such as 2% by weight of the composition.

The present composition may suitably be prepared by providing a solution of G hydroxyalkyl methylcellulose in a liquid diluent, optionally adding a surfactant to the solution as a processing aid. The active ingredient in powder or crystalline form and optionally one or more solid excipients (collectively termed “solids” herein) may then be mixed with the G hydroxyalkyl methylcellulose such that the weight ratio of G hydroxyalkyl methylcellulose solution to active ingredient is in the range of 0.1:1 to 0.85:1. The liquid diluent is preferably an aqueous liquid containing 50-100% water and may for instance be selected from purified water or water containing a surfactant acting as a defoaming aid during preparation of the composition. The weight ratio of liquid diluent to solids is preferably in the range of 0.1:1 to 0.75:1, 0.1:1 to 0.70:1, 0.1:1 to 0.65:1, 0.1:1 to 0.60:1, 0.1:1 to 0.60:1, 0.1:1 to 0.55:1, 0.1:1 to 0.50:1, 0.1:1 to 0.45:1 or 0.1:1 to 0.40:1.

Addition of a surfactant helps to distribute a low level of liquid diluent homogenously and produce a smooth highly viscous semi-solid paste, possibly due to defoaming and emulsification. The surfactant may be selected from conventional defoaming agents selected from the group consisting of anionic surfactants with anionic functional groups such as sulfates, sulfonates, phosphates and carboxylates such as alkyl sulfates, e.g. ammonium lauryl sulfate, sodium lauryl sulfate (sodium dodecyl sulfate, SLS, or SDS), and alkyl-ether sulfates, such as sodium laureth sulfate (sodium lauryl ether sulfate or SLES), and sodium myreth sulfate; cationic surfactants with cationic functional groups such as cetrimonium bromide (CTAB), cetylpyridinium chloride (CPC), benzalkonium chloride (BAC), benzethonium chloride (BZT), dimethyldioctadecylammonium chloride, dioctadecyldimethylammonium bromide (DODAB); zwitterionic surfactants such as cocamidopropyl betaine; and nonionic surfactants such as siloxane surfactants like modified polydimethylsiloxane-based defoamer, ethoxylates, fatty acid esters of glycerol, sorbitol and sucrose. The concentration of surfactant may be in the range of 0.1-1.5% by weight of the composition.

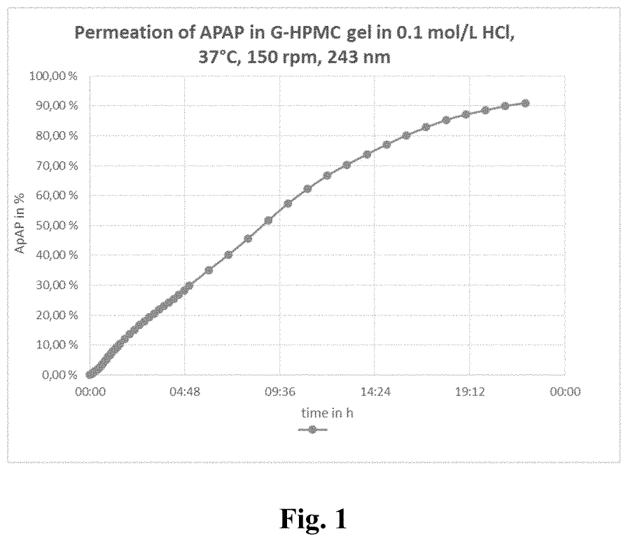

In one embodiment of the invention, the composition comprising G hydroxyalkyl methylcellulose admixed with the active ingredient is in the form of a dry powder. As appears from FIG. 1 appended hereto, a dried composition results in a slow release over time of the active ingredient under the conditions described in the examples below. The dry powder may be prepared by drying the mixture of the G hydroxyalkyl methylcellulose solution and active ingredient at a temperature of 40-100° C. until the mixture has a water content of less than 10% by weight, preferably less than 5% by weight, more preferably less than 3% by weight, in particular less than 2% by weight, such as less than 1% by weight, followed by milling or grinding the mixture to granules of a desired particle size in a manner known in the art. The dry powder will typically contain granules comprising the active ingredient partially or completely embedded in the G hydroxyalkyl methylcellulose which facilitates sustained release of the active ingredient as discussed above.

In one embodiment, the invention relates to a unit dosage form comprising the present composition. The unit dosage form is intended for oral administration and may be in the form of a tablet comprising compressed granules of the dried composition. Alternatively, the unit dosage form may be in the form of a tablet, granulate or pellet prepared by extruding the semi-solid paste prepared as described above and cutting the extruded mass into pieces of an appropriate size followed by drying. The tablet may optionally comprise one or more other excipients, though preferably G hydroxyalkyl methylcellulose is the only excipient included in the dosage form, except that a surfactant may optionally also be included as indicated above. The unit dosage form may also be a capsule including the dried composition, preferably in the form of dry granules containing the mixture of G hydroxyalkyl methylcellulose and active ingredient.

The unit dosage form contains one or more physiologically active ingredients, preferably one or more drugs, one or more diagnostic agents, or one or more physiologically active ingredients which are useful for cosmetic or nutritional purposes. The term “drug” denotes a compound having beneficial prophylactic and/or therapeutic properties when administered to an individual, typically a mammal, especially a human individual. The dosage form is believed to be particularly suited for administering highly dosed drugs, i.e. drugs administered in unit doses of 500 mg or more, as it is possible to provide a unit dose that includes the requisite amount of the active ingredient in a size that makes it easier to ingest. Examples of highly dosed drugs are metformin, metformin hydrochloride, acetaminophen (paracetamol) or acetylsalicylic acid. Thus, each unit dosage form may typically include 500-1000 mg of the active ingredient.

Some embodiments of the invention will now be described in detail in the following Examples.

Unless otherwise mentioned, all parts and percentages are by weight. In the Examples the following test procedures are used.

The determination of the % methoxyl and % hydroxypropoxyl in hydroxypropyl methylcellulose is carried out according to the United States Pharmacopeia (USP 32). The values obtained are % methoxyl and % hydroxypropoxyl. These are subsequently converted into degree of substitution (DS) for methyl substituents and molar substitution (MS) for hydroxypropyl substituents. Residual amounts of salt have been taken into account in the conversion.

The DS(methyl) and MS(hydroxyethyl) in hydroxyethyl methylcellulose is determined by Zeisel cleavage with hydrogen iodide followed by gas chromatography. (G. Bartelmus and R. Ketterer, Z. Anal. Chem. 286 (1977) 161-190).

Determination of s23/s26

The determination of ether substituents in cellulose ethers is generally known and e.g., described in Carbohydrate Research, 176 (1988) 137-144, Elsevier Science Publishers B.V., Amsterdam, DISTRIBUTION OF SUBSTITUENTS IN O-ETHYL-O-(2-HYDROXYETHYL)CELLULOSE by Bengt Lindberg, Ulf Lindquist, and Olle Stenberg.

Specifically, determination of s23/s26 is conducted as follows:

10-12 mg of the cellulose ether are dissolved in 4.0 mL of dry analytical grade dimethyl sulfoxide (DMSO) (Merck, Darmstadt, Germany, stored over 0.3 nm molecular sieve beads) at about 90° C. under stirring and then cooled down to room temperature again. The solution is left stirring at room temperature over night to ensure complete solubilization. The entire reaction including the solubilization of the cellulose ether is performed using a dry nitrogen atmosphere in a 4 mL screw cap vial. After solubilization the dissolved cellulose ether is transferred to a 22 mL screw cap vial. Powdered sodium hydroxide (freshly pestled, analytical grade, Merck, Darmstadt, Germany) and ethyl iodide (for synthesis, stabilized with silver, Merck-Schuchardt, Hohenbrunn, Germany) in a thirty fold molar excess of the reagents sodium hydroxide and ethyl iodide per hydroxyl group of the anhydroglucose unit are added and the solution is vigorously stirred under nitrogen in the dark for three days at ambient temperature. The perethylation is repeated with addition of the threefold amount of the reagents sodium hydroxide and ethyl iodide compared to the first reagent addition and further stirring at room temperature for additional two days. Optionally the reaction mixture can be diluted with up to 1.5 mL DMSO to ensure good mixing during the course of the reaction. 5 mL of 5% aqueous sodium thiosulfate solution is poured into the reaction mixture and the obtained solution is then extracted three times with 4 mL of dichloromethane. The combined extracts are washed three times with 2 mL of water. The organic phase is dried with anhydrous sodium sulfate (ca. 1 g). After filtration the solvent is removed in a gentle stream of nitrogen and the sample is stored at 4° C. until further sample preparation.

Hydrolysis of about 5 mg of the perethylated samples is performed under nitrogen in a 2 mL screw cap vial with 1 mL of 90% aqueous formic acid under stirring at 100° C. for 1 hour. The acid is removed in a stream of nitrogen at 35-40° C. and the hydrolysis is repeated with 1 mL of 2M aqueous trifluoroacetic acid for 3 hours at 120° C. in an inert nitrogen atmosphere under stirring. After completion the acid is removed to dryness in a stream of nitrogen at ambient temperature using ca. 1 mL of toluene for co-distillation.

The residues of the hydrolysis are reduced with 0.5 mL of 0.5 M sodium borodeuteride in 2N aqueous ammonia solution (freshly prepared) for 3 hours at room temperature under stirring. The excess reagent is destroyed by drop wise addition of ca. 200 μL of concentrated acetic acid. The resulting solution is evaporated to dryness in a stream of nitrogen at ca. 35-40° C. and subsequently dried in vacuum for 15 min at room temperature. The viscous residue is dissolved in 0.5 mL of 15% acetic acid in methanol and evaporated to dryness at room temperature. This is done five times and repeated four times with pure methanol. After the final evaporation the sample is dried in vacuum overnight at room temperature.

The residue of the reduction is acetylated with 600 μL of acetic anhydride and 150 μL of pyridine for 3 hrs at 90° C. After cooling the sample vial is filled with toluene and evaporated to dryness in a stream of nitrogen at room temperature. The residue is dissolved in 4 mL of dichloromethane and poured into 2 mL of water and extracted with 2 mL of dichloromethane. The extraction is repeated three times. The combined extracts are washed three times with 4 mL of water and dried with anhydrous sodium sulfate. The dried dichloromethane extract is subsequently submitted to GC analysis. Depending on the sensitivity of the GC system, a further dilution of the extract can be necessary.

Gas-liquid (GLC) chromatographic analyses are performed with Hewlett Packard 5890A and 5890A Series II type of gas chromatographs equipped with J&W capillary columns DB5, 30 m, 0.25 mm ID, 0.25 μm phase layer thickness operated with 1.5 bar helium carrier gas. The gas chromatograph is programmed with a temperature profile that holds constant at 60° C. for 1 min, heats up at a rate of 20° C./min to 200° C., heats further up with a rate of 4° C./min to 250° C., heats further up with a rate of 20° C./min to 310° C. where it is held constant for another 10 min. The injector temperature is set to 280° C. and the temperature of the flame ionization detector (FID) is set to 300° C. 1 μL of the samples is injected in the splitless mode at 0.5 min valve time. Data are acquired and processed with a Lab Systems Atlas work station.

Quantitative monomer composition data are obtained from the peak areas measured by GLC with FID detection. Molar responses of the monomers are calculated in line with the effective carbon number (ECN) concept but modified as described in the table below. The effective carbon number (ECN) concept has been described by Ackman (R. G. Ackman, J. Gas Chromatogr., 2 (1964) 173-179 and R. F. Addison, R. G. Ackman, J. Gas Chromatogr., 6 (1968) 135-138) and applied to the quantitative analysis of partially alkylated alditol acetates by Sweet et. al (D. P. Sweet, R. H. Shapiro, P. Albersheim, Carbohyd. Res., 40 (1975) 217-225).

ECN Increments Used for ECN Calculations:

| Type of carbon atom | ECN increment |
|---|---|
| hydrocarbon | 100 |
| primary alcohol | 55 |
| secondary alcohol | 45 |

In order to correct for the different molar responses of the monomers, the peak areas are multiplied by molar response factors MRFmonomer which are defined as the response relative to the 2,3,6-Me monomer. The 2,3,6-Me monomer is chosen as reference since it is present in all samples analyzed in the determination of s23/s26.

MRFmonomer=ECN2,3,6-Me/ECNmonomer

The mole fractions of the monomers are calculated by dividing the corrected peak areas by the total corrected peak area according to the following formulas:

s23=[(23-Me+23-Me-6-HAMe+23-Me-6-HA+23-Me-6-HAHAMe+23-Me-6-HAHA]; and

s26=[(26-Me+26-Me-3-HAMe+26-Me-3-HA+26-Me-3-HAHAMe+26-Me-3-HAHA], wherein

s23 is the sum of the molar fractions of anhydroglucose units which meet the following conditions:
a) the two hydroxy groups in the 2- and 3-positions of the anhydroglucose unit are substituted with methyl groups and the 6-position is not substituted (=23-Me);
b) the two hydroxy groups in the 2- and 3-positions of the anhydroglucose unit are substituted with methyl groups and the 6-position is substituted with methylated hydroxyalkyl (=23-Me-6-HAMe) or with a methylated side chain comprising 2 hydroxyalkyl groups (=23-Me-6-HAHAMe); and
c) the two hydroxy groups in the 2- and 3-positions of the anhydroglucose unit are substituted with methyl groups and the 6-position is substituted with hydroxyalkyl (=23-Me-6-HA) or with a side chain comprising 2 hydroxyalkyl groups (=23-Me-6-HAHA). s26 is the sum of the molar fractions of anhydroglucose units which meet the following conditions:
a) the two hydroxy groups in the 2- and 6-positions of the anhydroglucose unit are substituted with methyl groups and the 3-position is not substituted (=26-Me);
b) the two hydroxy groups in the 2- and 6-positions of the anhydroglucose unit are substituted with methyl groups and the 3-position is substituted with methylated hydroxyalkyl (=26-Me-3-HAMe) or with a methylated side chain comprising 2 hydroxyalkyl groups (=26-Me-3-HAHAMe); and
c) the two hydroxy groups in the 2- and 6-positions of the anhydroglucose unit are substituted with methyl groups and the 3-position is substituted with hydroxyalkyl (=26-Me-3-HA) or with a side chain comprising 2 hydroxyalkyl groups (=26-Me-3-HAHA).

The results of the determination of the substituents in the HAMC are listed in Table 4 below. In the case of HPMC's hydroxyalkyl (HA) is hydroxypropyl (HP) and methylated hydroxyalkyl (HAMe) is methylated hydroxypropyl (HPMe).

Production of a 2% Pure Aqueous Solution of the G-HPMC

To obtain a 2% aqueous solution of G-HPMC, 3 g of milled, ground, and dried G-HPMC (under consideration of the water content of the methylcellulose) were added to 147 g of tap water (temperature 20-25° C.) at room temperature while stirring with an overhead lab stirrer at 750 rpm with 3-wing (wing=2 cm) blade stirrer. The solution was then cooled to about 1.5° C. After the temperature of 1.5° C. was reached the solution was stirred for 180 min at 750 rpms. Prior to use or analysis, the solution was stirred for 15 min at 100 rpm in an ice bath.

Determination of the Viscosity of G-HPMC

The steady-shear-flow viscosity η(20° C., 10 s⁻¹, 2 wt. % MC) of an aqueous 2-wt. % G-HPMC solution was measured at 5° C. at a shear rate of 10 s⁻¹ with an Anton Paar Physica MCR 501 rheometer and cup and bob fixtures (CC-27).

Determination of Storage Modulus G′, Loss Modulus G″, Gelation Temperature t and Gel Strength

To characterize the temperature dependent properties of the precipitation or gelation of a 1.5 weight percent aqueous cellulose ether solution, an Anton Paar Physica MCR 501 rheometer (Ostfildern, Germany) with a Cup & Bob set-up (CC-27) and a peltier temperature control system is used in oscillation shear flow. These solutions are prepared according to the same dissolution procedure as described for the viscosity measurements. The measurements are performed at a constant frequency of 2 Hz. and a constant strain (deformation amplitude) of 0.5% from 10° C. to 85° C. with a heating rate of 1° C./min with a data collection rate of 4 points/min. The storage modulus G′, which is obtained from the oscillation measurements, represents the elastic properties of the solution. The loss modulus G″, which is obtained from the oscillation measurements, represents the viscous properties of the solution. At low temperature the loss modulus values G″ are higher than the storage modulus G′ and both values are slightly decreasing with increasing temperatures. If a precipitation takes places at elevated temperatures the storage modulus drops down. This precipitation temperature is analyzed from a plot of the log storage modulus G′ vs. temperature as the cross-over of two tangents. The first tangent is fitted to the decrease of the storage modulus with increasing temperatures and the second tangent is fitted to the drop of the storage modulus over a temperature region of 1-3° C. With further increasing temperatures the storage modulus values are increasing and a cross-over between the storage modulus and the loss modulus is obtained. The cross-over of G′ and G″ is determined to be the gelation temperature. Some cellulose ethers of the present invention might show two points of cross-over of G′ and G″. In such case the gelation temperature is the temperature at which G′/G″=1 and G″>G′ at a temperature which is 1° C. colder than G′/G″=1.

### Example 1: Preparation of G-HPMC

HPMC was produced according to the following procedure. Finely ground wood cellulose pulp was loaded into a jacketed, agitated reactor. The reactor was evacuated and purged with nitrogen to remove oxygen and then evacuated again. The reaction was carried out in two stages. In the first stage, a 50% by weight aqueous solution of sodium hydroxide was sprayed onto the cellulose in an amount of 1.2 moles of sodium hydroxide per mole of anhydroglucose units in the cellulose and the temperature was adjusted to 40° C. After stirring the mixture of aqueous sodium hydroxide solution and cellulose for about 30 minutes at 40° C., 1.5 moles of dimethyl ether, 3.5 moles of methylene chloride and 0.33 moles of propylene oxide per mole of anhydroglucose units were added to the reactor. The contents of the reactor were then heated in 60 min to 80° C. After having reached 80° C., the first stage reaction was allowed to proceed for 20 min.

Then a 50% by weight aqueous solution of sodium hydroxide at an amount of 1.0 moles of sodium hydroxide per mole of anhydroglucose units was added over a period of 90 min. The rate of addition was 0.011 moles of sodium hydroxide per mole of anhydroglucose units per minute. After the second stage addition was completed the contents of the reactor were kept at a temperature of 80° C. for 120 min.

After the reaction, the reactor was vented and cooled down to about 50° C. The contents of the reactor were removed and transferred to a tank containing hot water. The crude HPMC was then neutralized with formic acid and washed chloride free with hot water (assessed by AgNO₃ flocculation test), cooled to room temperature and dried at 55° C. in an air-swept drier. The material was then ground.

The resulting G-HPMC had a DS(methyl) of 1.50 and an MS(hydroxyalkyl) of 0.14, which corresponds to a methoxyl content of 24.3% and a hydroxypropoxyl content of 5.5%. The G-HPMC had a viscosity of 4890 MPa·s, measured as a 2 wt. % solution in water at 20° C. at a shear rate of 10 s¹, and a ratio s23/s26 of 0.18.

### Example 2: Release of Acetaminophen from Dried Gelatin Capsules Comprising G-HPMC

A 3% by weight aqueous solution of G-HPMC prepared as described in Example 1 was prepared and a modified polydimethylsiloxane-based defoamer (available from BASF under the trade name Foamstar SI2210) was added to the solution. 3.5 g of acetaminophen (abbreviated herein to APAP) was intimately mixed with 1.5 g of the solution of G-HPMC until a white homogenous and highly viscous paste was obtained. The content of Foamstar SI2210 in the paste was 0.115 g. The mixture was filled into a syringe and injected into gelatin capsules (size 000) which were subsequently closed and sealed. The mixture was carefully dried overnight at 50° C.

The dried capsules were placed in 900 ml of 0.1N HCl pH 1.1 at 37° C. and shaken at 150 rpm for 22 hours. Drug release was measured at a wavelength of 243 nm with a path length of 0.1 mm.

The release of APAP from the dried capsules is shown in FIG. 1 from which it appears that about 90% of the drug was released after 21 hours (shown as -●- in the figure).

### Example 3: Release of Acetaminophen from Gelatin Capsules Comprising K4M HPMC

A 2% by weight aqueous solution of METHOCEL™ K4M HPMC (available from DuPont) was prepared and a modified polydimethylsiloxane-based defoamer (available from BASF under the trade name Foamstar SI2210) was added to the solution. 9.75 g of acetaminophen (abbreviated herein to APAP) was intimately mixed with 5.25 g of the solution of METHOCEL™ K4M HPMC until a white homogenous and highly viscous paste was obtained. The content of Foamstar SI2210 in the paste was 0.115 g. The mixture was filled into a syringe and injected into gelatin capsules (size 000) which were subsequently closed. The filled capsules were immediately placed in 900 ml of 0.1N HCl pH 1.1 at 37° C. and shaken at 150 rpm for 50 hours. 250 μl samples were taken at intervals and analyzed for content of APAP.

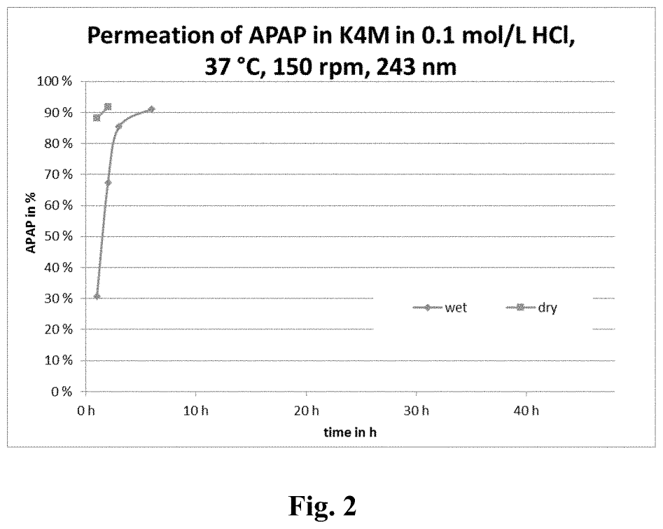

The release of APAP from the capsules is shown in FIG. 2 from which it appears that about 90% of the APAP was released from the capsules within 6 hours (shown as -♦- in the figure).

Gelatin capsules (size 000) were filled with about 1 g of the mixture and subsequently closed. The mixture was dried overnight at 50° C. The dried capsules were placed in 900 ml of 0.1N HCl pH 1.1 at 37° C. and shaken at 150 rpm for 22 hours. Drug release was measured at a wavelength of 243 nm with a path length of 0.1 mm.

The release of APAP from the capsules is shown in FIG. 2 from which it appears that about 90% of the drug was released after 1 hour (shown as -▪- in the figure). Thus, the rate of release from the dried capsules is faster than the rate of release from wet capsules.

Thus, neither the wet nor dried capsules that contained METHOCEL K4M HPMC as a matrix polymer provided sustained release of the active ingredient.

### Example 4: Release of Acetaminophen from Gelatin Capsules Comprising E4M HPMC

A 2% by weight aqueous solution of METHOCEL™ E4M HPMC (available from DuPont) was 9.75 g of acetaminophen (abbreviated herein to APAP) was intimately mixed with 5.25 g of the solution of METHOCEL™ E4M HPMC until a white homogenous and highly viscous paste was obtained. The mixture was filled into a syringe and injected into gelatin capsules (size 000) which were subsequently closed. The filled capsules were immediately placed in 900 ml of 0.1N HCl pH 1.1 at 37° C. and shaken at 150 rpm for 50 hours. 250 μl samples were taken at intervals and analyzed for content of APAP.

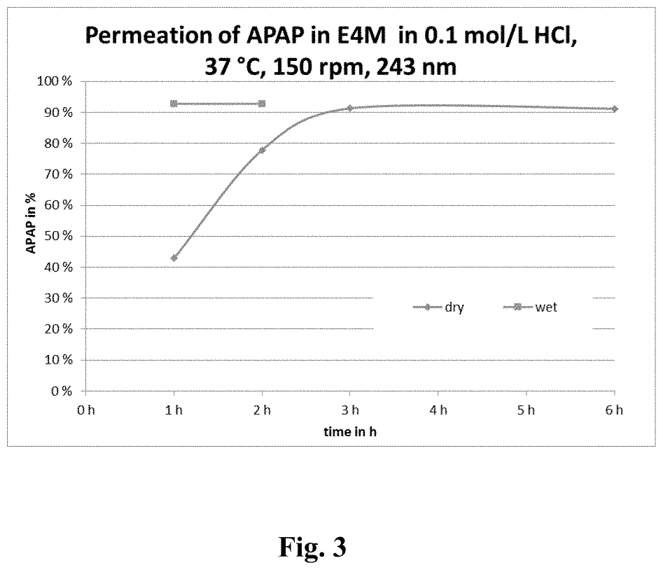

The release of APAP from the capsules is shown in FIG. 3 from which it appears that about 85% of the APAP was released from the capsules within 3 hours and that about 90% of the APAP was released from the capsules within 6 hours (shown as -♦- in the figure).

Gelatin capsules (size 000) were filled with about 1 g of the mixture and subsequently closed. The mixture was dried overnight at 50° C. The dried capsules were placed in 900 ml of 0.1N HCl pH 1.1 at 37° C. and shaken at 150 rpm for 22 hours. Drug release was measured at a wavelength of 243 nm with a path length of 0.1 mm.

The release of APAP from the capsules is shown in FIG. 3 from which it appears that about 90% of the drug was released after 1 hour (shown as -▪- in the figure). Thus, the rate of release from the dried capsules is faster than the rate of release from wet capsules.

Thus, neither the wet nor dried capsules that contained E4M HPMC as the matrix polymer provided sustained release of the active ingredient.

1. A sustained release composition for oral administration comprising particles of a physiologically active ingredient mixed with a hydroxyalkyl methylcellulose, wherein the ether substituents are methyl groups, hydroxyalkyl groups, and optionally alkyl groups being different from methyl,
the hydroxyalkyl methylcellulose has an MS(hydroxyalkyl) of 0.05 to 1.00, and
hydroxy groups of anhydroglucose units are substituted with methyl groups such that [s23/s26−0.2*MS(hydroxyalkyl)] is 0.30 or less,
wherein s23 is the molar fraction of anhydroglucose units wherein only the two hydroxy groups in the 2- and 3-positions of the anhydroglucose unit are substituted with methyl groups,
wherein s26 is the molar fraction of anhydroglucose units wherein only the two hydroxy groups in the 2- and 6-positions of the anhydroglucose unit are substituted with methyl groups, and
wherein the concentration of hydroxyalkyl methylcellulose is 0.1-10% by dry weight of the active ingredient.

2. The composition of claim 1, wherein the concentration of hydroxyalkyl methylcellulose is 0.2-5%, preferably 0.5-4%, more preferably 0.75-2% and still more preferably 1-1.8%, by dry weight of the active ingredient.

3. The composition of claim 1, wherein the concentration of hydroxyalkyl methylcellulose is about 1.5% by dry weight of the active ingredient.

4. The composition of claim 1 wherein the hydroxyalkyl methylcellulose is a hydroxypropyl methylcellulose and [s23/s26−0.2*MS(hydroxyalkyl)] is 0.27 or less.

5. The composition of claim 1, wherein the hydroxyalkyl methylcellulose is a hydroxyethyl methylcellulose and [s23/s26−0.2*MS(hydroxyalkyl)] is 0.30 or less.

6. The composition of claim 1, wherein the hydroxyalkyl methylcellulose has a DS(methyl) of 1.2 to 2.2.

7. The composition of claim 1, wherein the hydroxyalkyl methylcellulose constitutes at least 50%, preferably 60-100%, by weight of a polymeric matrix in which particles of the active ingredient are embedded.

8. The composition of claim 1 further comprising a surfactant.

9. The composition of claim 1, wherein the concentration of the surfactant is in the range of 0.1-1.5% by weight of the composition.

10. The composition of claim 1 further comprising an additive capable of reacting with gastric fluid to generate a gas.

11. The composition according to claim 10, wherein the additive is selected from alkali metal or alkaline earth metal carbonates, e.g. CaCO3 or Na2CO3.

12. The composition of claim 1 in the form of a dry powder.

13. A unit dosage form comprising a composition according to claim 1.

14. The unit dosage form of claim 13 comprising 500-1000 mg of the active ingredient.

15. The unit dosage form of claim 14, wherein the active ingredient is selected from the group consisting of metformin, metformin hydrochloride, acetaminophen and acetylsalicylic acid.
